(19) United States
(12) Patent Application Publication
(10) Pub. No.: US 2024/0066272 A1
(43) Pub. Date: Feb. 29, 2024

(54) Catheter Assembly with Distally-Directed Tube

(71) Applicant: Becton, Dickinson and Company, Franklin Lakes, NJ (US)

(72) Inventor: Jonathan Karl Burkholz, Salt Lake City, UT (US)

(21) Appl. No.: 18/238,601

(22) Filed: Aug. 28, 2023

Related U.S. Application Data
(60) Provisional application No. 63/401,907, filed on Aug. 29, 2022.

(51) Int. Cl. A61M 25/06; A61M 25/00; A61M 39/02; A61M 39/12; A61M 39/28

(52) U.S. Cl. CPC A61M 25/0606; A61M 25/0097; A61M 39/02; A61M 39/12; A61M 39/28; A61M 2025/0253

(57) ABSTRACT

Provided herein is a catheter assembly including a catheter adapter, having a distal end, a proximal end, a lumen extending between the distal end and the proximal end, and a side port arranged between the distal end and the proximal end, the side port in fluid communication with the lumen. The assembly further includes a catheter that is secured to the distal end of the catheter adapter and that is extending distally from the catheter adapter, a fluid conduit having a proximal end coupled to the side port and a distal end, the fluid conduit in fluid communication with the side port, and an elongated arm configured to extend distally away from the catheter adapter.

### CROSS-REFERENCE TO RELATED APPLICATION

The present application claims priority to U.S. Provisional Application Ser. No. 63/401,907 entitled “Catheter Assembly with Distally-Directed Tube” filed Aug. 29, 2022, the entire disclosure of which is hereby incorporated by reference in its entirety.

### BACKGROUND OF THE INVENTION

### Field of the Invention

Provided herein are devices and systems for use in vascular access.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations is the manipulation of the PIVC, for example, to manually connect various instruments thereto to enable blood draw; however, doing so in an uncontrolled way can increase incidence of dislodgement of the VAD from the vasculature, thus shortening the life of the catheter indwell. Accordingly, a need exists in the art for improved devices, systems, and methods of manipulating an indwelling catheter to free the catheter tip and allow for blood collection and fluid delivery therefrom or therethrough.

### SUMMARY OF THE INVENTION

Provided herein is a catheter assembly including a catheter adapter, having a distal end, a proximal end, a lumen extending between the distal end and the proximal end, and a side port arranged between the distal end and the proximal end, the side port in fluid communication with the lumen. The assembly further includes a catheter secured to the distal end of the catheter adapter and extending distally from the catheter adapter, a fluid conduit having a proximal end coupled to the side port and a distal end, the fluid conduit in fluid communication with the side port, and an elongated arm configured to extend distally away from the catheter adapter.

In accordance with an aspect of the present invention, a second elongated arm is coupled to the catheter adapter and configured to extend distally away from the catheter adapter.

In accordance with an aspect of the present invention, the elongated arms are arranged on opposite sides of the catheter adapter.

In accordance with an aspect of the present invention, the elongated arms are configured to intersect one another at a location distal to the distal end of the catheter adapter.

In accordance with an aspect of the present invention, the catheter assembly further includes an anchor configured to reversibly couple to the patient.

In accordance with an aspect of the present invention, the anchor is coupled to at least one of the elongated arms.

In accordance with an aspect of the present invention, the anchor is arranged at an intersection of the elongated arms.

In accordance with an aspect of the present invention, the catheter adapter includes a second side port arranged between the distal end and the proximal end, the side port in fluid communication with the lumen.

In accordance with an aspect of the present invention, the catheter assembly further includes a second fluid conduit coupled to and in fluid communication with the second side port.

In accordance with an aspect of the present invention, the catheter assembly further includes an anchor configured to reversibly couple to the patient.

In accordance with an aspect of the present invention, the anchor is coupled to the elongated arm.

In accordance with an aspect of the present invention, the first fluid conduit is configured to extend distally, and wherein the anchor is arranged at an intersection of the elongated arm and the first fluid conduit.

In accordance with an aspect of the present invention, the anchor is coupled to the first fluid conduit.

In accordance with an aspect of the present invention, the first fluid conduit is connected at a proximal end there to the catheter adapter, and wherein the first fluid conduit is configured to extend proximally from the catheter adapter.

In accordance with an aspect of the present invention, a connector is arranged at the distal end of the first fluid conduit.

In accordance with an aspect of the present invention, the connector includes a distal port coupled to the distal end of the first fluid conduit, a proximal port, a second lumen between the distal port and the proximal port, and a side port configured to be in fluid communication with the second lumen.

In accordance with an aspect of the present invention, a second fluid conduit having a proximal end is coupled to the connector side port and a distal end, the second fluid conduit in fluid communication with the connector side port, the second fluid conduit configured to extend distally from the connector.

In accordance with an aspect of the present invention, the elongated arm and the second fluid conduit are configured to intersect one another at a location distal to the catheter adapter.

In accordance with an aspect of the present invention, an anchor is configured to reversibly couple to the patient.

In accordance with an aspect of the present invention, the anchor is coupled to the second fluid conduit.

### BRIEF DESCRIPTION OF THE DRAWINGS

FIG. 1 is a perspective view of a non-limiting embodiment of a prior art catheter assembly useful with blood draw devices as described herein;

FIG. 2 is a top view of a non-limiting embodiment of a catheter assembly as described herein;

FIG. 3 is a top view of a non-limiting embodiment of a catheter assembly as described herein;

FIGS. 4A-4B are top views of non-limiting embodiments of a catheter assembly as described herein;

FIG. 5 is a top view of non-limiting embodiments of a catheter assembly as described herein;

FIGS. 6A-6B are top views of non-limiting embodiments of a catheter assembly as described herein; and

FIG. 7 is a top view of non-limiting embodiments of a catheter assembly as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

Provided herein are devices and systems for securing and/or stabilizing indwelling catheters, such as peripheral intravenous catheters (PIVCs). While certain devices (e.g., blood draw devices) are discussed below in terms of devices that may be used with PIVCs, as described herein, those of skill will appreciate that any number of different devices for introducing an instrument, including instruments ranging from tubes, probes, sensors, wiring, fiber optics, guidewires, etc., may be used within the scope of the present disclosure.

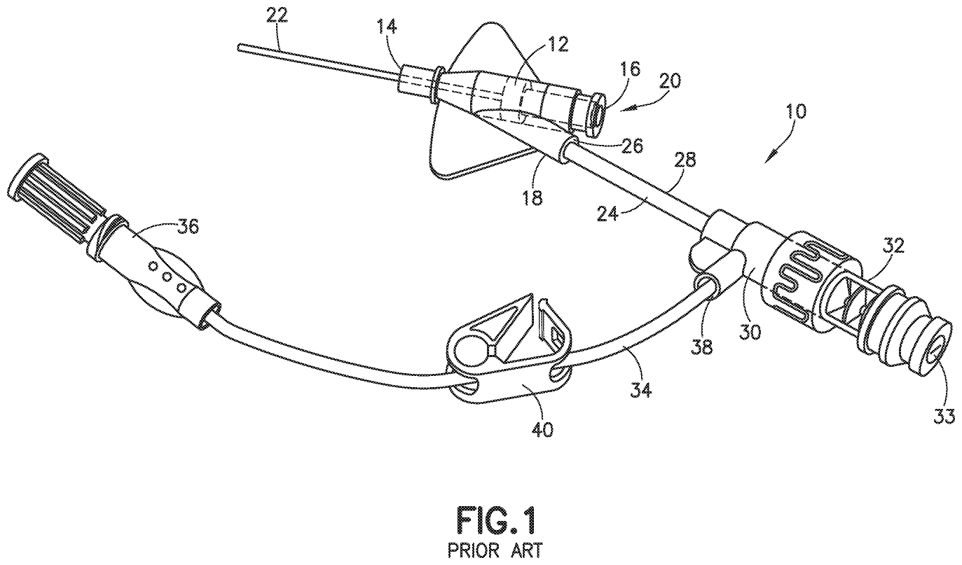

Referring now to FIG. 1, shown is a non-limiting embodiment of a prior art catheter assembly 10 useful with blood draw devices. Cather assembly 10 may include a catheter adapter 12, which may include a distal end 14 and a proximal end 16. In some embodiments, the catheter adapter 12 may include one or more additional ports 18. In some embodiments, port 18 may be disposed between the distal end 14 and the proximal end 16. In some embodiments, more than one port 18 may be disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 15 to 165 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

Catheter assembly 10 may include a needleless access connector 32 and/or a second fluid conduit 34. Suitable needleless access connectors 32 can include any split-septum connector and/or those with direct fluid path access. Needleless access connectors 32 are known to those of skill in the art and are commercially available from, for example, Becton, Dickinson and Company under the trade names Q-SYTE, and SMARTSITE. While the non-limiting embodiments of FIG. 1 show needleless access connectors arranged at connector 30, those of skill in the art will appreciate that suitable needleless access connectors may also be arranged at luer 36. In non-limiting embodiments, needleless access connector 32 includes a septum 33, such as a self-healing septum. In non-limiting embodiments, the septum 33 is a slit-type septum. As will be described below, a blood draw device, as described herein, may be reversibly coupleable to the needleless access connector 32, and one or more portions of the blood draw device may pierce the septum 33.

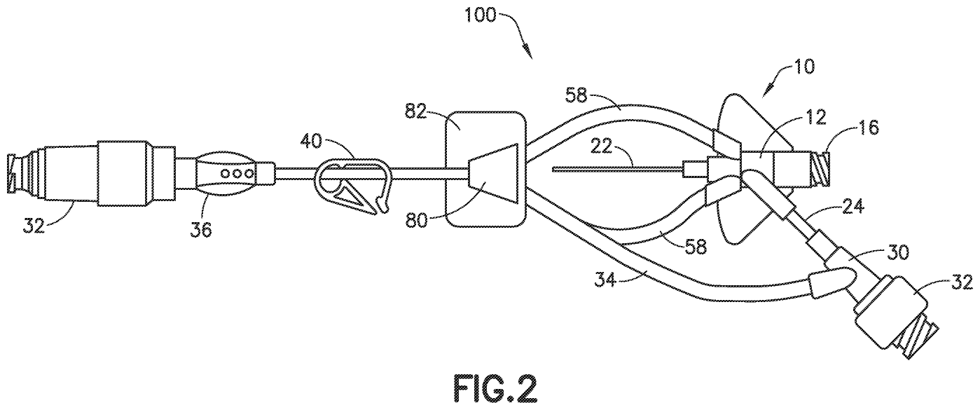

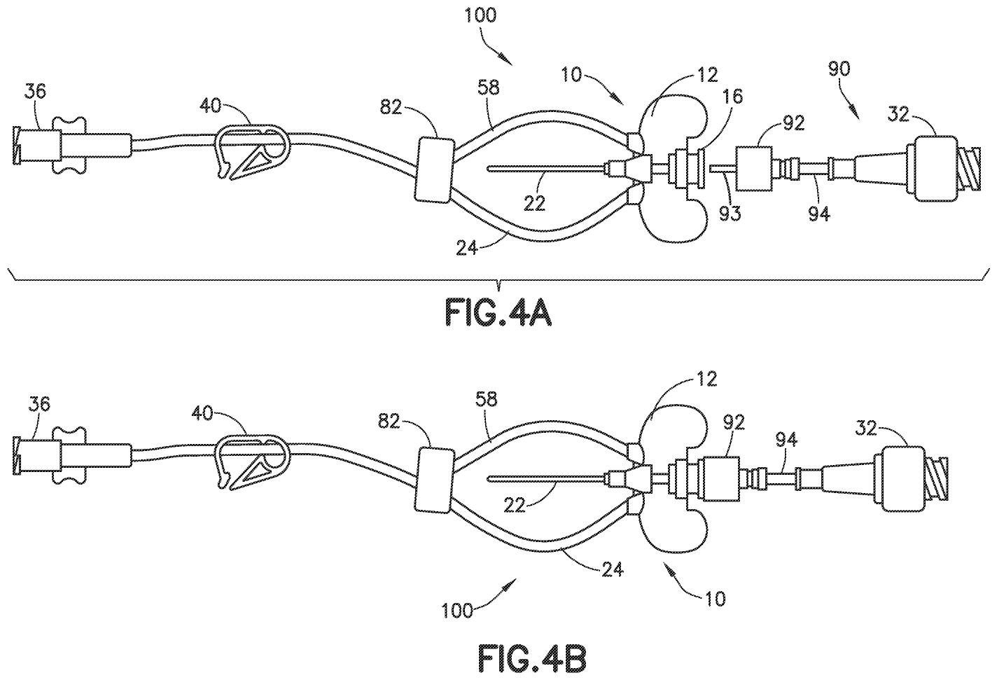

Turning to FIGS. 2-7, shown are non-limiting embodiments of a catheter assembly 100 including a catheter adapter 12 (optionally with a stabilization platform), a fluid conduit 34, and one or more elongated arms 58. One or more elongated arms 58 may be coupled to catheter adapter 12. Fluid conduit 34 and/or one or more arms 58 may be arranged on opposite sides of catheter adapter 12, and may be configured to extend distally (for example, as shown in FIGS. 4A-4B, 6A-6B, and 7) from catheter adapter 12. In non-limiting embodiments, fluid conduit 34 and one or more elongated arms 58 extend distally along an arc, thereby intersecting distally of catheter adapter 12. At a point where fluid conduit 34 and one or more arms 58 intersect, a joint 80 and/or an anchor 82 may be arranged, to couple fluid conduit 34 and one or more elongated arms 58. Joint 80 and anchor 82 may be arranged at different locations, for example, joint 80 may be arranged at an intersection of one or more elongated arms 58 and/or fluid conduits, and anchor 82 may be arranged distally, for example, only coupled to a fluid conduit. Fluid conduit 34 may include, at a distal end thereof, a connector, such as a luer connecter 36 and/or a needle-free connector (NFC) (also known as a needless access connector, NAC) 32. Those of skill will appreciate that NFCs/NACs 32, as described herein, may be integrated into conduits and/or connectors, or may be removably coupled to such structures. Catheter assembly 100 may further include a clamp 40, arrangeable anywhere along the length of fluid conduit 34.

Catheter assembly 100 may be coupled to an instrument delivery device or other component through proximal end 16. As shown in FIGS. 4A-4B, catheter assembly 100 may be coupled to a further port, by coupling proximal end 16 of catheter adapter 12 to luer adapter 92. Luer adapter 92 may be coupled to a fluid conduit 94, which may have at an end thereof a NFC/NAC 32.

One or more elongated arms 58 may be formed of any suitable material. While not wishing to be bound by the theory, elongated arm(s) 58 with a material having a substantially similar elasticity to materials typically used in fluid conduits, such as fluid conduit 34, may be preferable to minimize twisting of catheter adapter 12 when a distally-directed force is applied to one or more elongated arms 58 and/or fluid conduit 34. By arranging one or more elongated arms 58 and/or fluid conduit 34 in the manner shown in the accompanying figures, force can be applied to catheter adapter 12 and, consequently, catheter 22, whereby catheter 22 is pulled further into the vasculature, rather than inadvertently being pulled out of the vasculature if, for example, fluid conduit 34 extended proximally.

The non-limiting embodiments of FIGS. 2-7 show various possible configurations of catheter assembly 100, but those of skill will appreciate that these should not be considered limiting. For example, in non-limiting embodiments, elongated arm(s) 58 may also be fluid conduits, coupled to one or more side ports 18 of catheter adapter 12. In non-limiting embodiments, catheter assembly 100 includes one or two elongated arms 58, one or more fluid conduits 34, and various combinations thereof.

With continuing reference to the accompanying figures, anchor 82 may be configured to be reversibly coupleable to the patient. For example, anchor 82 may include a backing on a skin-facing surface that includes an adhesive. Suitable adhesives for reversibly coupling components to a patient's skin are known to those of skill in the art. Anchor 82 may be formed of any suitable material, for example, flexible materials, soft materials, semi-rigid materials, or rigid materials. Anchor 82 may also include one or more visible indicia, conveying information to healthcare professionals, such as catheter gauge, length, and/or information relating to one or more procedures to be performed through catheter assembly 100.

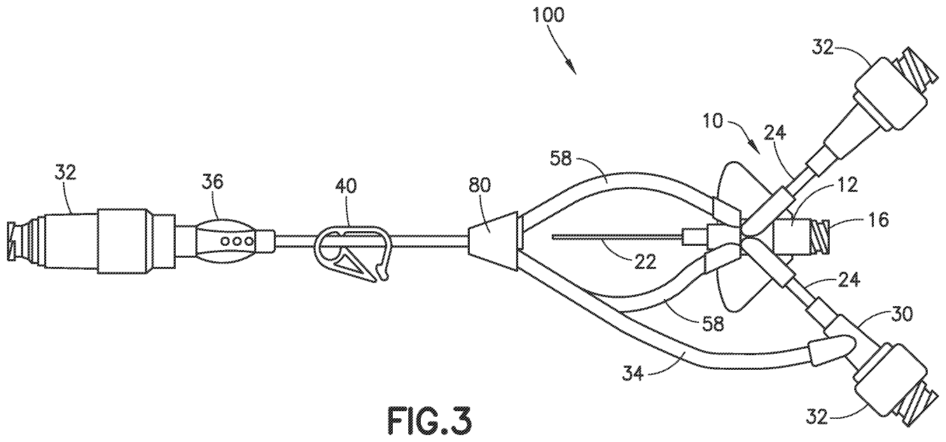

Turning to FIGS. 2 and 3, shown are non-limiting embodiments of catheter assembly 100, in which a plurality of elongated arms 58 are coupled to catheter adapter 12 and extend distally therefrom, and one (FIG. 2) or two (FIG. 3) proximally-directed side ports 18 are included. In both figures, a first fluid conduit 24 is coupled to one of the side ports, and has, at a distal end thereof, a connector 30 (which may include a NFC/NAC 32), the connector including a side port to which a second fluid conduit 34 is coupled. Second fluid conduit 34 is distally extending, and may, as shown in these non-limiting examples, intersect with elongated arms 58 at junction 80, which is arranged with anchor 82. In the illustrated non-limiting embodiments, a clamp 40 is included on second fluid conduit 34, and at distal end of second fluid conduit 34 a luer 36 with a NFC/NAC 32 is arranged. In the non-limiting embodiment of FIG. 3, catheter adapter 12 includes a plurality of side ports 18, and a further first fluid conduit 24 is coupled to another side port. This further first fluid conduit 24 includes, at a distal end thereof, a NFC/NAC 32. Those of skill will appreciate that luers 36 and/or NFCs/NACs 32 in the embodiments of FIGS. 2 and 3, as well as any other embodiment discussed herein, may be replaced with any other suitable structure and/or connector.

Turning to FIGS. 4A-4B, shown are non-limiting embodiments of catheter assembly 100, prior to (FIG. 4A) and after (FIG. 4B) coupling of a near-patient access port (here, a NFC/NAC 32). The near-patient access port includes a luer 92 for coupling to catheter adapter 12, a cannula 93, and a fluid conduit 94. In the illustrated non-limiting embodiment, catheter assembly 100 includes only a single elongated arm 58, and first fluid conduit 24 extends distally from side port 18, intersecting with elongated arm 58 at anchor 82. In the illustrated non-limiting embodiments, a clamp 40 is included on fluid conduit 24, and at distal end of fluid conduit 24 a luer 36 is arranged.

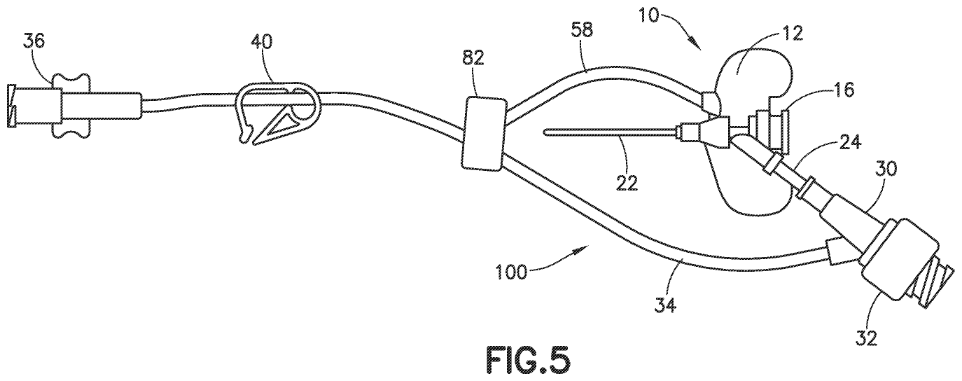

Turning to FIG. 5, shown is a non-limiting embodiment of catheter assembly 100 in which a single elongated arm 58 is coupled to catheter adapter 12 and extends distally therefrom, and one proximally-directed side port 18 is included. First fluid conduit 24 is coupled to the side port 18, and has, at a distal end thereof, a connector 30 (which may include a NFC/NAC 32), the connector including a side port to which second fluid conduit 34 is coupled. Second fluid conduit 34 is distally extending, and may, as shown in these non-limiting examples, intersect with elongated arm 58 at anchor 82. In the illustrated non-limiting embodiments, a clamp 40 is included on second fluid conduit 34, and at distal end of second fluid conduit 34 a luer 36 is arranged.

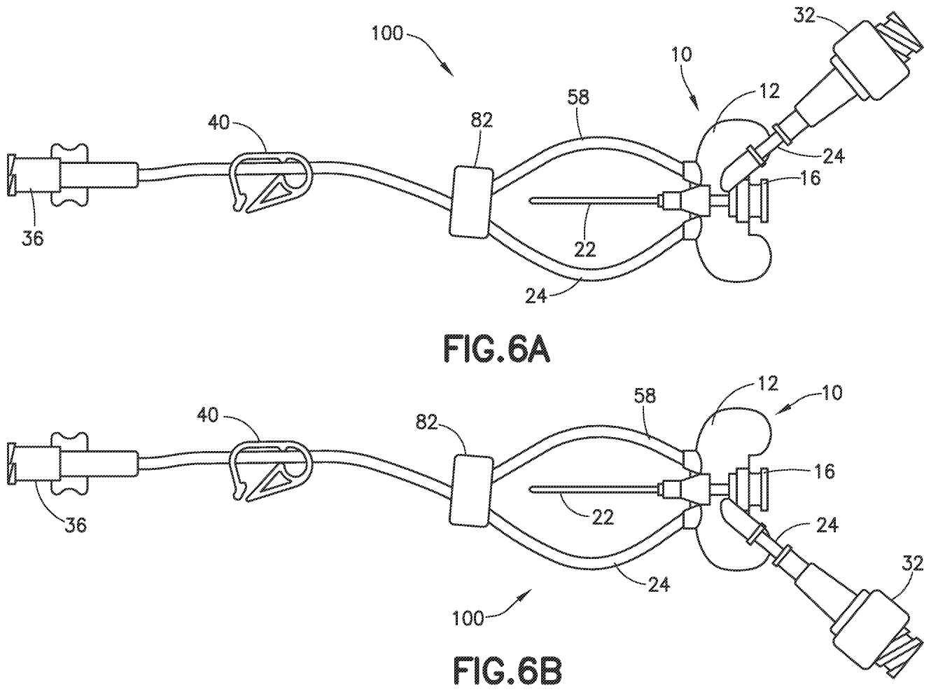

Turning to FIGS. 6A-6B, shown are non-limiting embodiments of catheter assembly 100. In the illustrated non-limiting embodiments, catheter assembly 100 includes only a single elongated arm 58, and first fluid conduit 24 extends distally from side port 18, intersecting with elongated arm 58 at anchor 82. In the illustrated non-limiting embodiments, a clamp 40 is included on fluid conduit 24, and at distal end of fluid conduit 24 a luer 36 is arranged. In the illustrated non-limiting embodiments, catheter adapter 12 further includes a proximally-directed side port (on the opposite side (FIG. 5A) or the same side (FIG. 5B)), as the distally-directed side port to which first fluid conduit 24 is coupled), to which a further first fluid conduit 24 is coupled. This further first conduit 24 includes, at a distal end thereof, a NFC/NAC 32.

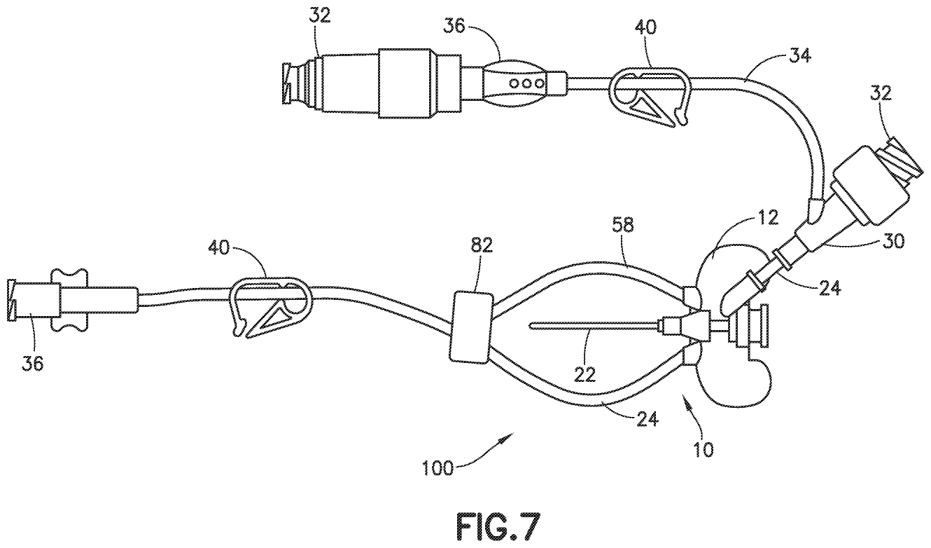

Turning to FIG. 7, shown is a non-limiting embodiment of catheter assembly 100. In the illustrated non-limiting embodiment, catheter assembly 100 includes only a single elongated arm 58, and first fluid conduit 24 extends distally from side port 18, intersecting with elongated arm 58 at anchor 82. In the illustrated non-limiting embodiment, a clamp 40 is included on fluid conduit 24, and at distal end of fluid conduit 24 a luer 36 is arranged. In the illustrated non-limiting embodiment, catheter adapter 12 further includes a proximally-directed side port (on the opposite side, though those of skill will appreciate that other arrangements are possible, as the distally-directed side port to which first fluid conduit 24 is coupled), to which a further first fluid conduit 24 is coupled. This further first conduit 24 includes, at a distal end thereof, a connector 30, which includes, as an integral or removable component, a NFC/NAC 32. Connector 30 includes a side port to which a second fluid conduit 34 is coupled. In the illustrated non-limiting embodiment, a clamp 40 is included on second fluid conduit 34, and at distal end of second fluid conduit 34 a luer 36 with a NFC/NAC 32 is arranged.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter assembly comprising:
a catheter adapter comprising:
a distal end;
a proximal end;
a lumen extending between the distal end and the proximal end; and
a side port arranged between the distal end and the proximal end, the side port in fluid communication with the lumen;
a catheter secured to the distal end of the catheter adapter and extending distally from the catheter adapter;
a first fluid conduit having a proximal end coupled to the side port and a distal end, the first fluid conduit in fluid communication with the side port; and
an elongated arm configured to extend distally away from the catheter adapter.

2. The catheter assembly of claim 1, further comprising a second elongated arm coupled to the catheter adapter and configured to extend distally away from the catheter adapter.

3. The catheter assembly of claim 2, wherein the elongated arms are arranged on opposite sides of the catheter adapter.

4. The catheter assembly of claim 2, wherein the elongated arms are configured to intersect one another at a location distal to the distal end of the catheter adapter.

5. The catheter assembly of claim 4, further comprising an anchor configured to reversibly couple to the patient.

6. The catheter assembly of claim 5, wherein the anchor is coupled to at least one of the elongated arms.

7. The catheter assembly of claim 5, wherein the anchor is arranged at an intersection of the elongated arms.

8. The catheter assembly of claim 1, wherein the catheter adapter comprises a second side port arranged between the distal end and the proximal end, the side port in fluid communication with the lumen.

9. The catheter assembly of claim 8, further comprising a second fluid conduit coupled to and in fluid communication with the second side port.

10. The catheter assembly of claim 1, further comprising an anchor configured to reversibly couple to the patient.

11. The catheter assembly of claim 10, wherein the anchor is coupled to the elongated arm.

12. The catheter assembly of claim 10, wherein the first fluid conduit is configured to extend distally, and wherein the anchor is arranged at an intersection of the elongated arm and the first fluid conduit.

13. The catheter assembly of claim 10, wherein the anchor is coupled to the first fluid conduit.

14. The catheter assembly of claim 1, wherein the first fluid conduit is connected at a proximal end there to the catheter adapter, and wherein the first fluid conduit is configured to extend proximally from the catheter adapter.

15. The catheter assembly of claim 14, further comprising a connector arranged at the distal end of the first fluid conduit.

16. The catheter assembly of claim 15, wherein the connector comprises a distal port coupled to the distal end of the first fluid conduit, a proximal port, a second lumen between the distal port and the proximal port, and a side port configured to be in fluid communication with the second lumen.

17. The catheter assembly of claim 16, further comprising a second fluid conduit having a proximal end coupled to the connector side port and a distal end, the second fluid conduit in fluid communication with the connector side port, the second fluid conduit configured to extend distally from the connector.

18. The catheter assembly of claim 17, wherein the elongated arm and the second fluid conduit are configured to intersect one another at a location distal to the catheter adapter.

19. The catheter assembly of claim 18, further comprising an anchor configured to reversibly couple to the patient.

20. The catheter assembly of claim 19, wherein the anchor is coupled to the second fluid conduit.
